(19) United States
(12) Patent Application Publication
(10) Pub. No.: US 2023/0131447 A1
(43) Pub. Date: Apr. 27, 2023

(54) A TWO-WAY VACUUM ASSISTED CLOSURE SYSTEM

(71) Applicants: Seracettin EGIN, Istanbul (TR); Semih HOT, Kucukcekmece, Istanbul (TR)

(72) Inventors: Seracettin EGIN, Istanbul (TR); Semih HOT, Kucukcekmece, Istanbul (TR)

(21) Appl. No.: 17/047,590

(22) PCT Filed: Oct. 11, 2019
(86) PCT No.: PCT/TR2019/050860

§ 371 Date: Oct. 14, 2020

(30) Foreign Application Priority Data
May 10, 2019 (TR) 2019/07076

(51) Int. Cl. A61F 13/14; A61M 1/00

(52) U.S. Cl. CPC A61F 13/148; A61M 1/915; A61M 1/916; A61M 1/917; A61M 1/966; A61M 2210/1021

(57) ABSTRACT

Disclosed is a two-way vacuum assisted closure system in the medical field which eliminates leakages based on the upper gastrointestinal system organs such as the stomach and intestine after stab wounds and/or wounds after surgical operations.

### TECHNICAL FIELD

The invention is related to a closure system which eliminates the leakages based on the upper gastrointestinal system organs such as stomach and intestine after the stab wounds and/or wounds after surgical operations.

The invention is particularly related to a two-way vacuum assisted closure system which is used in treating the leakages in general surgery operation complications, in the leakages occurred after the operations made between the region where the esophagus passes through the muscle of the diaphragm and the stomach, small intestine and large intestine, in the leakages occurred after the stomach is joined with the duodenum or the small intestine, in the leakages occurred after the repairs due to stab wounds and firearm injuries of the region where the esophagus passes through the muscle of the diaphragm, the stomach and duodenum, in the leakages occurred after joining the biliary tracts with the duodenum and the small intestine, in the leakages occurred after the repairs due to perforation of the stomach and duodenum ulcer.

### STATE OF THE ART

Today in clinical applications various complications may be seen;
during the surgical operations made between the region where the esophagus passes through the muscle of the diaphragm and the stomach, small intestine and large intestine,
after small intestine and large intestine operations,
during the operations of joining the stomach with the duodenum and the small intestine,
during the operations for repairing the stab wounds and firearm injuries of the stomach and the duodenum,
during the operations of joining biliary tracts with the duodenum and small intestine,
after treatment operations of stomach ulcer and duodenum ulcer

Examples to these complications are the leakages experienced on the operation region. For said complications first of all in order to apply stent endoscopically to the anatomical regions which create leakage and to discharge the accumulation of the leakage within the abdominal cavity outside, it is taken out of the body by means of a needle or a hose placed under the guidance of radiological imaging methods. However, when these processes are insufficient, many subsequent operations must be made. Death ratio and additional surgical complication ratio increases in the patients that are subject to subsequent operations. In the repeated operations in case said leakage is not treated, advanced level peritonitis arising from a perforation on the digestive tract or an infection in the abdominal cavity, sepsis arising out of the mixture of bacteria or toxin to the blood and is known as blood poisoning, septic shock which generally occurs as a result of the distribution of an infection generally in the localized form in the beginning in the blood and multi organ failure are developed and death is an inevitable result in these patients.

In the current applications, in case the leakage is in the duodenum whose anatomy is degraded after particularly duodenum operations, stent applications are not possible in this region. In order to limit the duodenum leakage within the region, surgical operations which try to limit the leakage region by omentum is performed and passive drainage operations are performed by means of drains in order to remove the leakages accumulated in the bottom portion of the liver. The success ratio in this limitation and passive drainage processes are low according to the surgical literature.

In the leakages occurred after the operations of the upper gastrointestinal systems namely the stomach and intestine, what protects the patients from sepsis is the complete control of the primary source. Contamination of the abdominal cavity by means of the intestine content in a continuous manner must be certainly prevented. In case the continuous contamination of the abdominal cavity by means of the intestine content loaded by the bacteria cannot be prevented, death is inevitable due to sepsis of the patient. The main rule for recovery of these patients is to make a complete control of the primary source. In the current applications, the main source control is very difficult in the leakages after the upper gastrointestinal region surgery and mostly it is not possible in a complete manner.

As a result of the research made in terms of the subject matter, we come across with the application with publication No EP2968016B1 EPC titled as “Negative pressure wound closure device and systems and methods of use in treating wounds with negative pressure”. In case the open abdomen is closed by means of said apparatus in a temporary manner, the amount of the negative pressure applied to the center of open abdomen by means of the connection hose decreases at the wall of the abdomen region. The pressure measurement in terms of this apparatus is given as 71 mmHg on the internal organ protective sponge arm on the farthermost end within the wall when 88 mmHg negative pressures is applied at the center. Although the pressure does not decrease on the wall in an explicit manner at the alignment of the arms with sponge, the wall pressure in the region between the arms with sponge, which is spongeless and where only internal organs are under protection, decreases to 15 mmHg. Due to explicit low negative pressure between the arms of this sponge, the leakages accumulate in the abdomen chamber at the level of the spongeless regions. In order to activate the immune system properly, the small intestine content, which is loaded with an important substance namely cytokine, exudates which is a fluid accumulated after an inflammatory process and close to the serum value and transudes which are far from serum value cannot be effectively discharged out of the abdomen. On the wall at side portions of the abdomen on which 15 mmHg pressure is affective, toxic leakages accumulate which is harmful for the patient. Also, on the abdomen wall where low pressure (15 mmHg) is applied, due to low negative pressure, the development rate of the granulation tissue decreases. The development rate of the granulation tissue increases in a directly proportional manner together with the increase of the small vascular circulation namely micro vascular circulation by the effect of the negative pressure. Due to this scientific reality, due to a decrease in the development rate of the granulation tissue in the low negative pressure region, its recovery is not facilitated with the granulation tissue of the damaged tissue.

As a result, due to the above-mentioned disadvantages and insufficiency of the current solutions about the subject matter, an improvement is required to be made in the relevant technical field.

### Aim of the Invention

The invention aims to solve the above-mentioned disadvantages by means of being inspired from the present conditions.

In virtue of the invention, after the upper digestion system surgery, in the leakages into the abdomen without the requirement of secondary surgical interventions, by means of applying open abdomen technique, the abdomen is closed in a temporary manner with the utility model which is named as the two-way vacuum assisted closure, thus active drainage of the toxic fluids flowing into the abdomen of the patient is possible by means of a shorter way. Therefore, before the toxic fluids flowing into the abdominal cavity distribute totally, development of peritonitis and sepsis is prevented.

Another aim of the invention is to allow active drainage process of the toxic fluids flowing into the abdomen region of the patient by means of a shorter way, by closing the abdomen region in a temporary manner.

Another aim of the invention is to allow discharging the localized leakages out of the abdomen in a shorter, active, effective and fast manner in the leakages based on the operations of the region where the esophagus passes through the diaphragm muscle and stomach operations and small-large intestine operations, by means of the additional sponge extension.

Another aim of the invention is to place one end of the additional sponge arm on the immediate surface or side of the damaged tissue that causes leakage, the other end of it is removed out of the abdomen from the nearest side wall to the damaged tissue.

Another aim of the invention is to increase the granulation speed.

The structural and characteristic features of the present invention will be understood clearly by the following drawings and the detailed description made with reference to these drawings and therefore the evaluation shall be made by taking these figures and the detailed description into consideration.

### FIGURES CLARIFYING THE INVENTION

FIG. 1 is a demounted view of the inventive the two-way vacuum assisted closure system elements.

FIG. 2 is a view of the inventive the two-way vacuum assisted closure system which is applied on the patient.

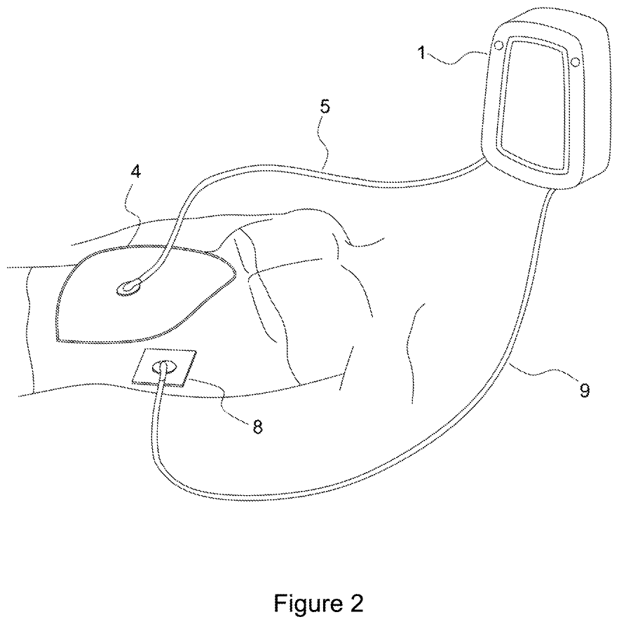

### DESCRIPTION OF PART REFERENCES

1. Vacuum device
2. Organ protective sponge
3. Elliptical sponge
4. Adhesive cover
5. Connection hose
6. Organ protective additional sponge
7. Organ non-protective additional sponge
8. Additional adhesive cover
9. Additional connection hose

### DETAILED DESCRIPTION OF THE INVENTION

In this detailed description, preferred embodiments of the inventive the two-way vacuum assisted closure system are described only for clarifying the subject matter.

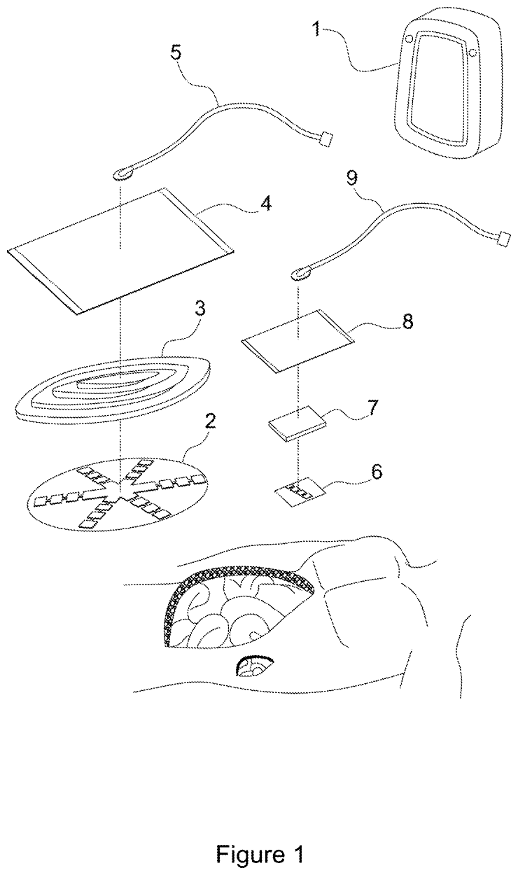

The vacuum device (1) which enables vacuum process seen in FIG. 1, which is chargeable and operates with electric, enables continuous or intermittent negative pressure.

6-armed organ protective sponge (2) is placed up to the grooves next to the colon on both sides within the abdomen. Both surfaces of the organ protective sponge (2) are covered with double-layer internal organ protective layers which are made of aromatic thermoplastic polyurethanes. On these organ protective layers, there are micropores changing between 400 to 600 micron lengths for the passage of the fluids. The organ protective sponge (2) transfers the cytokine loaded fluids within the abdomen to the elliptical sponge (3) by means of absorbing thereof with the micropores.

The elliptical sponge (3) which has an elliptical shape applied on the organ protective sponge (2), is a complementary sponge layer for the temporary abdomen closure which is at the same level with the skin level, transfers the fluids coming from the organ protective sponge (2) layer to the connection hose (5). The bottom surface of the elliptical sponge (3) is brought to the same level with the abdomen connective tissue; upper surface is brought to the same level with the skin. Also, because the elliptical sponge (3) does not have any contact with the internal organs, the internal organ is not required to be covered by the protective layer.

The adhesive cover (4) prevents air entrance by means of sticking to the elliptical sponge (3) and on the skin of the front wall of the abdomen on both sides of the open abdomen. The adhesive cover (4) provides a closed negative pressure by means of cutting the contact with the atmosphere totally.

The connection hose (5), transfers the negative pressure, which is provided by the vacuum device (1), to the elliptical sponge (3) and the organ protective sponge (2). There are pressure sensors within the connection hose (5) and the applied pressure is measured by means of these pressure sensors.

The organ protective additional sponge (6) has a similar structure alike the organ protective sponge (2) and has one arm. Both surfaces are covered with double layer internal organ protective layers made of aromatic thermoplastic polyurethane. On these organ protective layers, there are micropores changing between 400 to 600 micron lengths for the passage of the fluids. It transfers the leakages coming from the damaged organ to the organ non-protective additional sponge (7) which is located on the skin at the abdomen side wall in a shorter manner.

The organ non-protective additional sponge (7) is a sponge which has a rectangular shape applied on the organ protective additional sponge (6) which is removed out of the abdomen side wall and does not have an organ protective cover on it. It transfers the fluids coming through the organ protective additional sponge (6) to the additional connection hose (9).

The additional adhesive cover (8) prevents air entrance by means of sticking onto the organ protective additional sponge (6) which is removed out of the abdomen side wall and onto the abdomen side wall skin. The additional adhesive cover (8) thus provides a closed negative pressure environment by means of passing its contact with the atmosphere totally.

The additional connection hose (9) transfers the negative pressure which is provided by the vacuum device (1) to the organ protective additional sponge (6) removed out of the abdomen side wall. There are pressure sensors within the additional connection hose (9) and the value of the applied pressure is measured by means of these pressure sensors.

In the invention, one end of the organ protective additional sponge (6) which is added to the current technique is placed immediate onto or next the surface of the damaged tissue which causes leakage and the other end is removed out of the closest abdomen side wall to the damaged tissue. 50-100 mmHg negative pressure is applied to the end out of the abdomen. Preferred pressure value is generally 75 mmHg. Since the organ protective additional sponge (6) is short, it is transmitted to the end of the organ protective additional sponge (6) which is in contact with the damaged tissue at the applied negative pressure value. The small intestine content loaded with cytokines leaked from the damaged tissue is taken outside in a shorter manner by means of a 75 mmHg negative pressure value without the accumulation in the abdomen. The leakages never contaminate the other sections of the abdomen. The remaining portion of the abdomen out of the region where there is leakage remains clean. Peritonitis development is not possible since leakage contamination is not present. The product of the current technique applied on the central portion of the abdomen operates in a continuous mode in a manner such that there is a negative pressure with a second collecting hose. The negative pressure value applied on the central portion is 75-125 mmHg. Together with 75 mmHg pressure applied immediate onto and next to the damaged tissue with the organ protective additional sponge (6), since the micro vascular circulation is increased in the damaged tissue in an effective manner granulation develops in a faster manner and the damaged tissue which causes leakage is recovered by means of the granulation tissue in a fast manner.

Usage of the Closure System Subject to the Invention;

After the open abdomen is washed and cleaned by means of a warm physiological saline solution, in the leakages occurred in the region where the esophagus passes through the diaphragm muscle, the organ protective additional sponge (6) which is placed on the left bottom portion of the diaphragm muscle in a manner such that it comes next to this region is take out of the abdomen from the left side wall of the abdomen and bottom portion of the costa spring. The other end of the organ protective additional sponge (6), is taken out of the abdomen from the left side wall of the abdomen and bottom portion of the costa spring in the leakages between the region where the esophagus passes through the diaphragm muscle and the stomach, is taken out of the abdomen from the abdomen right side wall and bottom portion of the costa spring in the leakages of duodenum. The organ protective sponge (2) is placed up to the combined grooves with the colon at sides in the open abdomen. The elliptical sponge (3) is at the connective tissue level at the bottom and at the skin level at the upper portion, together with the adhesive cover (4), the organ non-protective elliptical sponge (3) and the abdomen front wall skin on both sides of the cut made to the abdominal region are covered. The organ non-protective additional sponge (7) is placed onto the end of the organ protective additional sponge (6) which is removed out of the abdomen in a manner such that there remains an organ protective layer between the skin and the organ protective additional sponge (6) is pasted on the skin by means of the additional adhesive cover (8). The circular gap with 2-3 cm diameter on the adhesive cover (4) on the middle portion of the open abdomen can only be formed by means of cutting the adhesive cover (4). A connection hose (5) is pasted onto this gap with a circular shape. The connection hose (5) is connected with the vacuum device (1). The circular form gap with 2-3 cm on the additional adhesive cover (8) corresponds on the organ non-protective additional sponge (7) can only be formed by means of cutting the additional adhesive cover (8). An additional connection hose (9) is pasted onto this circular form gap and the additional connection hose (9) is connected to the vacuum device (1). The vacuum device is operated by means of adjusting 75-125 mmHg negative pressure values.

1. A two-way vacuum assisted closure system which eliminates the leakages based on organs in the surgical operation complications, comprising:
a vacuum device which enables vacuuming process with negative pressure;
an organ protective sponge which will be placed up to the grooves in a manner such that it is joined with the colon within the abdomen and transfers the fluids accumulated herein;
an elliptical sponge which is placed onto the organ protective sponge and transfers the absorbed fluids received from the organ protective sponge;
an adhesive cover which prevents air entrance by means of sticking onto the elliptical sponge and onto the abdomen front wall skin on both sides of the open abdomen and enables a closed negative pressure environment by means of cutting the atmosphere contact;
a connection hose which is pasted onto the adhesive cover, transfers the negative pressure provided by the vacuum device to the elliptical sponge and the organ protective sponge and transfers the gathered fluids;
characterized by further comprising:
an organ protective additional sponge whose one end is placed onto or next to the damaged tissue causing leakage and other end is removed out of the closest side wall to the damaged tissue, transfers the leakages by means of gathering the leakages coming through the damaged organs;
an organ non-protective additional sponge which is placed on the organ protective additional sponge and transfers the fluids accumulated on the organ protective additional sponge;
an additional adhesive cover which prevents air entrance within the abdomen by sticking onto the organ protective additional sponge which is removed out of the abdomen side wall and onto abdomen side wall skin and provides a negative atmosphere pressure by means of cutting the atmosphere contact; and
an additional connection hose which is pasted on the additional adhesive cover and transmits the negative pressure provided by the vacuum device to the organ non-protective additional sponge and the organ protective additional sponge and transfers the gathered fluids.

2. The two-way vacuum assisted closure system according to claim 1, comprising a six-arm organ protective sponge.

3. The two-way vacuum assisted closure system according to claim 1, comprising a single-arm organ protective additional sponge.

4. The two-way vacuum assisted closure system according to claim 1, comprising an armless organ non-protective additional sponge.

5. The two-way vacuum assisted closure system according to claim 1, comprising micropores on the organ protective additional sponge for the passage of the fluids.

6. The two-way vacuum assisted closure system according to claim 1, comprising a pressure value sensor measuring pressure of the additional connection hose.
